(19) United States
(12) Patent Application Publication
(10) Pub. No.: US 2011/0092780 A1
(43) Pub. Date: Apr. 21, 2011

(54) BIOSENSOR MODULE WITH AUTOMATIC POWER ON CAPABILITY

(76) Inventors: Tao Zhang, Natick, MA (US); Oliver Orion Wilder-Smith, Holliston, MA (US)

(21) Appl. No.: 12/840,074

(22) Filed: Jul. 20, 2010

Related U.S. Application Data
(60) Provisional application No. 61/252,337, filed on Oct. 16, 2009.

(51) Int. Cl. A61B 5/053; A61B 5/01; A61B 5/11

(52) U.S. Cl. 600/301; 600/547

(57) ABSTRACT

A biosensor is described which can obtain physiological and accelerometer data from an individual. The biosensor may collect electrodermal activity, accelerometer readings, skin temperature, and other information. Most of the biosensor may be powered off when it is not attached to a person. Based on electrodermal activity the biosensor may automatically turn on when the biosensor comes in contact with an individual. The biosensor may rapidly power up once placed on a person even being fully functional within one second of being attached.

### RELATED APPLICATIONS

This application claims the benefit of the U.S. provisional patent application Ser. No. 61/252,337 filed Oct. 16, 2009 “Biosensor module” which is hereby incorporated by reference in its entirety.

### FIELD OF INVENTION

This application relates generally to biosensor modules and more particularly to automatically powering on biosensor modules.

### BACKGROUND

Physiological and other information on individuals can be extremely useful when evaluating health and activity. Physiological information may include electrodermal activity, also known as skin conductance. Physiological information may further include evaluation of skin temperature, heart rate, heart rate variability, and other aspects of the human body's operation. Useful information may also be found through tracking movements such as those which may be collected through accelerometer readings. All these readings and other information may be collected to evaluate the health of an individual, to diagnose numerous health problems, and to track physical or exercise activity.

Further, the physiological and other data collected can be useful in evaluating health or other information on an individual. Various states of being active, including the motions of gesturing, can be evaluated by tracking accelerometer readings. Many of the physiological readings and other information may be obtained through a biosensor attached to a human body. Biosensors have been either stationary or portable. Historically these biosensors, however, have been cumbersome and difficult to use. The presence of a cumbersome biosensor could even impact the user's readings, simply by the awareness of the person to the biosensor.

There remains a need for improved monitoring of physiological and other information through improved biosensor modules.

### SUMMARY

Analysis of physiological readings from a person can be key in evaluating health or even the mental state of an individual. A biosensor may be provided to monitor motion and physiological readings for an individual.

A wearable apparatus is disclosed for monitoring physiological information for an individual comprising: a plurality of electrodes for contacting skin on the individual; a battery powering at least one of the plurality of electrodes; a sensor for determining skin conductance across the plurality of electrodes; and a power control that responds to the sensor wherein the sensor determines that the plurality of electrodes are in contact with the skin based on the skin conductance and wherein the power control powers up circuitry when the plurality of electrodes are in contact with the skin. The power control may include a voltage regulator. The power control may include clock gating. The power control may include disabling a microcontroller. The plurality of electrodes may comprise two electrodes. The sensor may include an analog sensing stage to measure conductance between the two electrodes in contact with the skin and wherein the sensor further includes a high-gain transconductance amplifier circuit. The conductance which is measured may comprise a current value. The sensor may further comprise an operational amplifier connected to one of the plurality of electrodes wherein the operational amplifier is used to evaluate the skin conductance. The sensor may further comprise a comparator which determines that a voltage on an output of the operational amplifier has risen above a threshold. The voltage regulator may turn on within 5 seconds of the plurality of electrodes contacting the skin. Real time clock circuitry may be connected to the battery. A microcontroller, a temperature sensor, an accelerometer, and a storage memory may each have its power controlled by the voltage regulator.

In some embodiments, a method for powering on a wearable physiological sensor may comprise: contacting skin of an individual to a pair of electrodes; sensing skin conductance for the individual through the pair of electrodes; and turning on an output of a voltage regulator in response to the sensing of the skin conductance based on the contacting of the skin to the pair of electrodes. The sensing may be accomplished by an analog sensing stage to measure current between the pair of electrodes in contact with the skin and wherein the analog sensing stage further includes a high-gain transconductance amplifier circuit. Real time clock circuitry may not be connected to the output of the voltage regulator. A microcontroller, a temperature sensor, an accelerometer, and a storage memory may each be connected to the output of the voltage regulator.

In some embodiments, a wearable system for detecting physiological information from an individual may comprise: a pair of electrodes for contacting skin of the individual; power supply means for powering at least one of the pair of electrodes; sensing means for determining skin conductance across the pair of electrodes; and a voltage regulator that responds to the sensing means wherein the sensing means determines that the pair of electrodes is in contact with the skin based on the skin conductance and wherein the voltage regulator powers up circuitry when the pair of electrodes are in contact with the skin. The real time clock circuitry may be connected to the power supply means.

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a block diagram of a biosensor module.

FIGS. 2A & B are schematics of a biosensor.

FIG. 3 is a layout of the top of a sensor card.

FIG. 4 is a layout of the bottom of a sensor card.

FIG. 5 is a flowchart for automatically powering on physiological sensor.

FIG. 6A is a diagram of a biosensor attached to an individual.

FIG. 6B is a drawing of a biosensor with a wristband.

### DETAILED DESCRIPTION

The present disclosure provides a description of various apparatus, methods, and systems associated with sensing physiological and other information related to an individual. There is a need for an improved sensor of this type of information. A biosensor may obtain information on electrodermal activity, skin temperature, accelerometer readings, heart rate, heart rate variability, blood pressure, blood sugar, and other information about an individual. The collected information may be used to monitor the health of an individual. The collected information may be used to monitor and evaluate the mental state of the individual. Monitoring such mental states can be useful for both therapeutic and business purposes. Mental states run a broad gamut from happiness to sadness, from contentedness to worry, from excitement to calmness, as well as numerous others. Many mental states, such as engagement, excitement, confusion, concentration, and worry, may be identified to aid in the understanding of an individual and their response to certain stimuli such as an advertisement, walking through a store, interacting with a web site, a movie, a movie trailer, a product, a computer game, a video game, or consuming a food.

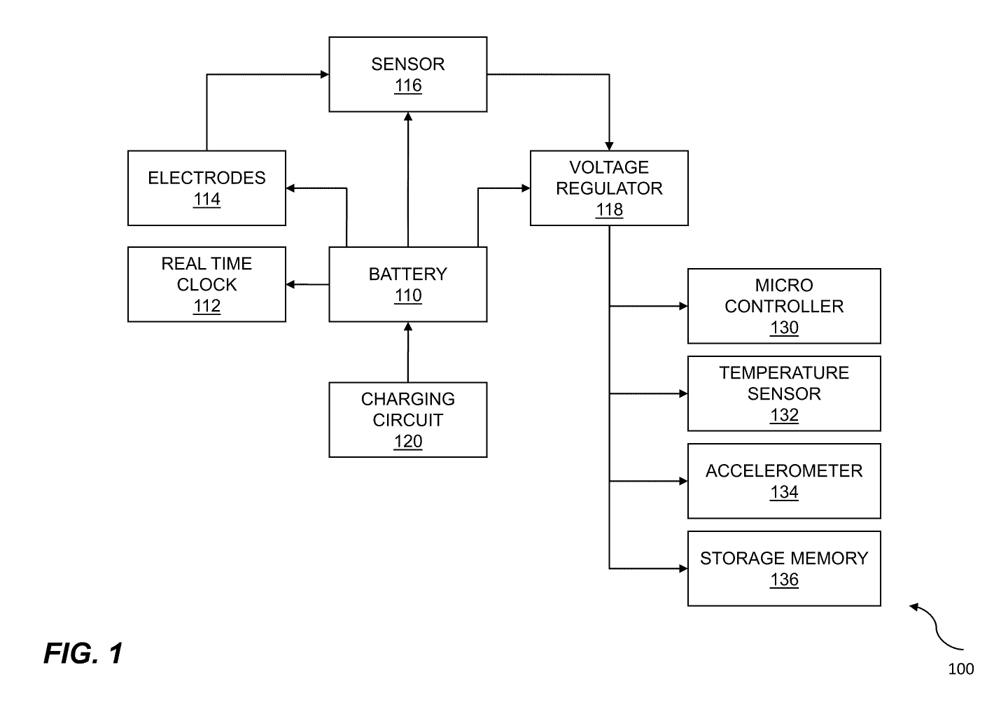

FIG. 1 a block diagram of a biosensor module 100. A battery 110 may be connected to a real time clock 112, electrodes 114, a sensor 116, and a voltage regulator 118. The battery 110 may be chargeable or may be replaceable. The battery 110 may be charged through a charging circuit 120. The battery 110 may be rechargeable through the charging circuit 120 using a universal serial bus (USB) connection to the biosensor module 100. The battery 110 may range in value from 3.6 V to 4.2 V depending on the amount of charge in the battery 110. The USB connection may be a standard USB connection, a mini-USB, a micro-USB, or some other type of connection. The charging circuit 120 may sense a connection to a power source through a USB or other connection using a diode configuration allowing current to flow from the USB port into the battery 110 being charged.

The real time clock 112 may keep track of time and may be set through a USB port connection. The real time clock circuitry 112 may be connected to the battery 110. By having the battery 110 connected to the real time clock 112, the clock is able to maintain the proper time even when most of the biosensor module 100 is powered down. The real time clock 112 may use a crystal to help the clock keep proper time.

There may be a plurality of electrodes 114. In some embodiments, the plurality of electrodes 114 may comprise two electrodes. The battery 110 may be connected to one or more electrodes 114. In some embodiments the battery 110 may be connected to a single electrode. Current may be conducted from that electrode to a second electrode. The second electrode may be connected to a sensor circuit 116. This sensor circuit 116 may be connected to the battery 110 so that the sensor may remain active even when most of the biosensor module 100 is powered down. The sensor 116 may evaluate when current flows between a pair of electrodes thereby indicating that skin is in contact with the electrodes and thus there is electrodermal activity. The electrodermal activity may also be known as skin conductance. When skin is in contact with the electrodes 114, the sensor 116 may sense skin conductance. The sensor 116 may be connected to a voltage regulator 118. The sensor 116, having sensed the electrodes 114 being in contact with skin, may turn on the voltage regulator 118.

When the voltage regulator 118 turns on, the output may provide power to the remainder of the biosensor module 100. The microcontroller 130, the temperature sensor 132, the accelerometer 134, and the storage memory 136 may each have its power controlled by the voltage regulator 118. The voltage regulator 118 may provide voltage to a microcontroller 130, a temperature sensor 132, an accelerometer 134, and storage memory 136. The microcontroller 130 may collect and analyze information collected from the biosensor module 100. The microcontroller 130 may also control various configuration aspects of the biosensor. These configurations may be accomplished without removal of the biosensor from the individual. An algorithm may be stored on the biosensor, through firmware update or otherwise, which determines a context of operation and then updates the configuration of the biosensor automatically. The temperature sensor 132 may sense the temperature of the skin surface. The skin surface temperature may be evaluated through one or more of the electrodes 114. The temperature sensor 132 may evaluate the temperature of part or all of the biosensor module 100. Under certain circumstances the temperature sensor 132 may sense an elevated temperature and in communication with other parts of the biosensor module 100 shut down the power for all or part of the biosensor module. In some embodiments, the voltage regulator 118 may be turned off so that the portion of the biosensor module 100 which is controlled by the voltage regulator 118 may be powered down.

The accelerometer 134 may sense motion for the individual wearing the biosensor module 100. The accelerometer 134 may include detection of motion in three axes, e.g. x, y, and z axis motion. The accelerometer 134 may be used to detect motion associated with gesturing, physical exercise, sleeping, and the like.

The storage memory 136 may be used for storing instructions that may be executed on the micro controller 130. Likewise, the storage memory 136 may be used for storing data or status from the micro controller 130. The storage memory 136 may be non-volatile memory such as Flash, magnetoresistive random access memory (MRAM), ferroelectric random access memory (FeRAM), or phase change memory. The storage memory 136 may also include some volatile memory such as SRAM or DRAM. The storage memory 136 may be used to store configuration information for the biosensor module 100. The configuration information may be used to define the sampling rate of the various physiological and accelerometer readings. The configuration information may be used to turn off one or more light emitting diodes (LEDs). These LEDs may be turned off during a period of sleep for the individual so that the light does not disturb their sleep cycle. A context of operation may be an activity in which an individual is involved. By understanding the context of operation, such as a person being asleep, resting, gesturing, being active, or physically exercising, a different sampling rate may be chosen because beneficial. For example, if a person is resting, the sampling rate for electrodermal activity may be decreased. In another example if a person is sleeping, the sampling rate for electrodermal activity may be increased in order to detect a transition in sleep stages. In another example, if a person is gesturing, the sampling rate of the accelerometer might be increased in order to detect greater agitation, for instance.

In some embodiments when the biosensor module 100 is removed, the electrodes 114 lose contact with the skin. The sensor 116 may sense that the contact with the skin has been lost and the voltage regulator may be turned off in order to disconnect power from the remainder of the biosensor module 100 and thereby save power and extend the life of the battery 110. In some embodiments, motion detected with the accelerometer 134 may be used to evaluate if the biosensor module 100 is still being worn and thereby be used in determining whether to turn off the output of the voltage regulator 118. In some embodiments, when no movement is detected by the accelerometer 134 and little electrodermal activity is detected by the sensor 114, the voltage regulator 116 may be turned off.

The biosensor 100 may be a wearable apparatus for monitoring physiological information for an individual comprising a plurality of electrodes 114 for contacting skin on the individual, a battery 110 powering at least one of the plurality of electrodes 114, a sensor 116 for determining skin conductance across the plurality of electrodes 114, and a power control that responds to the sensor 116 wherein the sensor 116 determines that the plurality of electrodes 114 are in contact with the skin based on the skin conductance and wherein the power control powers up circuitry when the electrodes 114 are in contact with the skin. The power control may include a voltage regulator 118 where the voltage regulator may reduce the supply voltage to a portion of the biosensor 100 circuitry. The reduced voltage may be zero volts or some other number which significantly reduces the power consumption of the circuitry to which the reduced voltage is supplied. The power control may include clock gating. Portions of the circuitry for the biosensor 100 may have a regular clock signal which is supplied to the circuitry. For instance a clock signal may be supplied to the microcontroller 130. When the power control gates a clock signal to the microcontroller 130, for instance, the microcontroller 130 holds it state and does not continue to process data which is sent to it. In this manner clock gating may reduce the power consumed by the microcontroller 130 or other circuitry to which the clock is gated. The power control may include disabling the microcontroller 130. In some embodiments the power control may send a signal to the microcontroller 130. When the microcontroller 130 detects this signal an instruction may be processed which prevents further processing by parts of the microcontroller 130. For instance a data cache or other circuitry on the microcontroller 130 may be prevented from being updated and thereby power consumption may be decreased.

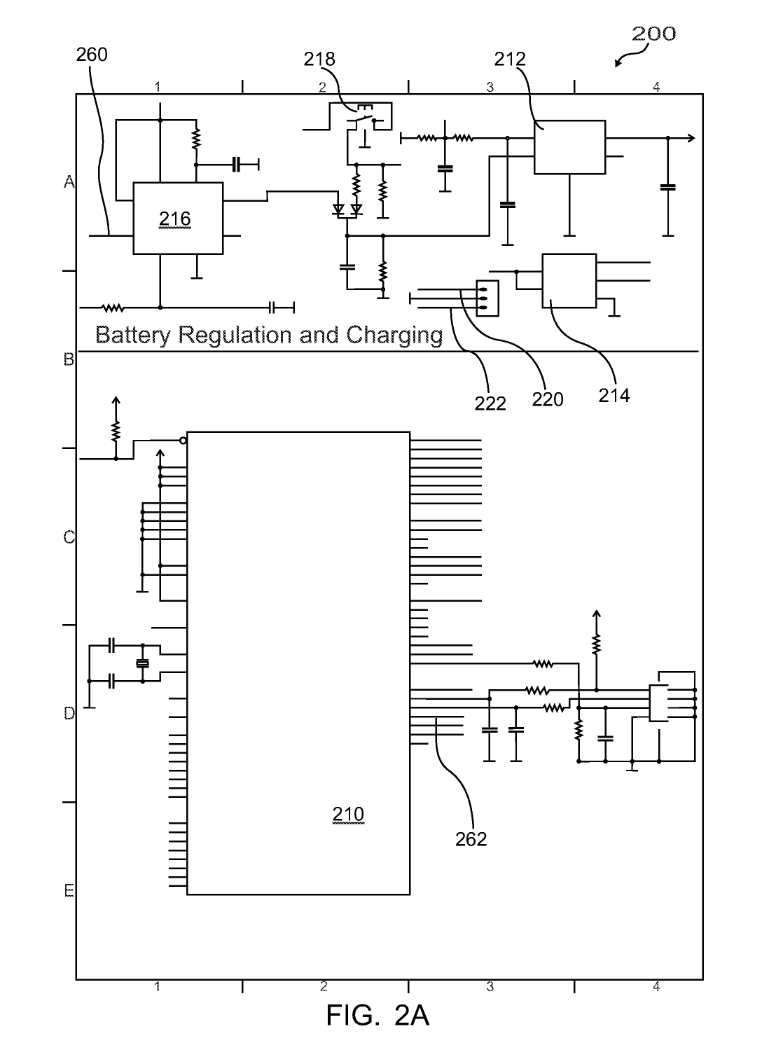

FIGS. 2A & B are schematics of a biosensor 200. FIG. 2A shows the microcontroller along with battery regulation and charging. The microcontroller 210 may be used to analyze data which is collected and manage the biosensor 200.

A voltage regulator 212 may be used to control power to part of the biosensor 200 in order to reduce power consumption during times when the biosensor 200 may be inactive. The voltage regulator 212 may be connected to a battery (not shown) and may gate voltage to part of the biosensor 200 in order to extend battery life. A charger 214 may be used to charge the battery. The charger 214 may be connected to a USB port and power to charge the battery may come from this USB port. A flip flop 216 may be used to store state for the biosensor. When a flip flop output is in a “1” state the biosensor 200 may be powered on. When the flip flop output is in a “0” state the biosensor 200 may be powered off. The flip flop 216 may be connected to the voltage regulator 212 so that the state of the flip flop 216 turns on the voltage regulator 212. The flip flop 216 may be turned on by the biosensor 200 sensing electrodermal activity when the biosensor 200 comes in contact with skin of an individual. In some embodiments, a button 218 may be used to turn on the voltage regulator 212. By depressing the button 218 the flip flop 216 may be set to the “1” state.

The biosensor 200 may include a plurality of electrodes. These electrodes may be considered “snaps” where the snaps may be snapped into the biosensor 200. The snaps may therefore be replaced as desired. A first electrode 220 may be connected to the battery. A second electrode 222 may be connected to a sensing circuit 250 shown in FIG. 2B. Electrodermal activity may be detected by having current flow from the first electrode 220 to the second electrode 222. A battery or other device may be used as a power supply to the biosensor 200. The power supply may include a wired connection, such as through a USB or other port, a capacitor, or some other electromechanical provision of power to the biosensor 200.

FIG. 2B shows parts of the biosensor 200 including the sensor circuit 240, the real time clock 250, the accelerometer 252, the temperature sensor 254, and LEDs 256. The sensor circuit 240 may be connected to the second electrode 222. The second electrode 222 may be connected to a first operational amplifier (OpAmp) 242. The output of the first OpAmp 242 may be connected to an input of a second OpAmp 244. The output of the second OpAmp 244 may be an input to the first OpAmp 242. The output of the second OpAmp 244 may also be an input to a comparator 246. The other input to the comparator 246 may be a resistor divider network which provides a threshold voltage. When the output of the second OpAmp 244 is higher than the threshold voltage the sensor determines that electrodermal activity has been sensed due to the electrodes being in contact with skin of the individual. A comparator output 260 goes to a “1” state when this electrodermal activity is sensed. The comparator output 260 may be connected to the flip flop 216 so that the flip flop output may be set to a “1” state and the voltage regulator 212 may be turned on. The sensor 240 may comprise a comparator 246 which determines that a voltage on an output of the operational amplifier 244 has risen above a threshold. The sensor circuit 240 may also have an analog output 262 providing the electrodermal activity value. This analog output 262 may be connected to the microcontroller 210 so that the electrodermal activity value may be analyzed by the biosensor 200. A second analog signal from the sensor 240 may also be connected to the microcontroller 210 as part of the electrodermal activity analysis by the biosensor 200. The sensor 240 may include an analog sensing stage to measure conductance between the pair of electrodes in contact with the skin and wherein the sensor may further include a high-gain transconductance amplifier circuit. The conductance which is measured may comprise a current value. The sensor 240 may further comprise an operational amplifier 242 connected to one of the plurality of electrodes 222 wherein the operational amplifier 242 is used to evaluate the skin conductance. The sensing may be accomplished by the analog sensing stage.

The real time clock 250 may be connected to a battery so that the real time clock 250 may keep time even when the voltage regulator 212 has its output powered off. The real time clock 250 provides input to the microcontroller 210 so that times may be associated with data which the biosensor 200 collects. The accelerometer 252 may detect motion in three axes. Data collected from the accelerometer 252 may be provided as input to the microcontroller 210. The accelerometer may be powered by the output of the voltage regulator 212. The temperature sensor 254 may evaluate the temperature of the skin on the individual to which the biosensor 200 is attached. The temperature sensor 254 may provide the temperature data to the microcontroller 210. The temperature sensor 254 may also detect over temperature conditions for the biosensor 200. Detection of an over temperature condition may allow the biosensor to power down to prevent damage to the biosensor 200 or to prevent harm to the individual. The temperature sensor 254 may be powered by the output of the voltage regulator 212. LEDs 256 may provide indication of operation for the biosensor 200. When the biosensor 200 turns on a light emitting diode (LED) may blink. As operation continues an LED may blink occasionally. The LEDs 256 may be powered by the output of the voltage regulator 212. The LEDs may be turned off when the individual is asleep in order to not disturb the individual. In some embodiments, the color of the LED may change based on the mental state of the individual. In some embodiments, the color or intensity or number of LEDs being lit may change based on the amount of electrodermal activity.

Sampling rates for the various pieces of information detected by the biosensor 200 may be modified. These sampling rates may all be modified together or may be separately controlled. For instance a sampling rate may be determined for evaluating the electrodermal activity. This sampling rate may be eight times per second. In some situations the sampling rate may be modified up to 32 times per second or may be modified down to 2 times per second. In some cases a sampling rate of 128 times per second may be desirous and therefore be implemented with the biosensor 200. Higher and lower sampling rates are possible for various situations. In some embodiments, sampling rate may be changed based on the mental state of the individual. The situations could be based on context where context is used to mean the state of the individual. A context might be that the individual is sleeping. Another context might be that the individual is gesturing. Based on the readings of the accelerometer 252, an individual could be determined to be gesturing. The sampling rate for obtaining information from the accelerometer 252, the temperature sensor 254, and the sensor 240 evaluating electrodermal activity could be modified so that all sample at 32 times per second. Alternatively, the various information types being sampled by the biosensor 200 could each have different sampling rates. If the individual is active or resting or sleeping, the sampling rate could be varied as most appropriate for the information being obtained given the specific context. Information on the sampling rate and base time may be stored along with the information being sampled. By storing all this information, the absolute time may be determined for each piece of information sampled. When this information is downloaded for further analysis, the times, events, and information sampled can all be reconstructed to provide a synchronized perspective of the data sampled.

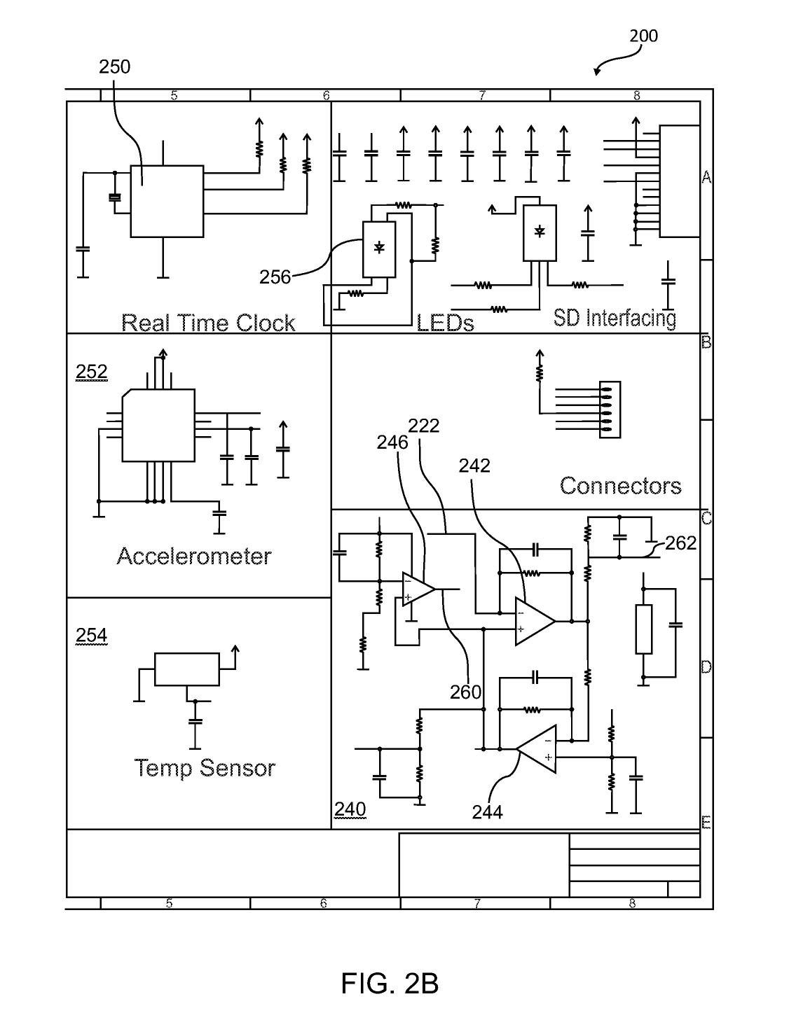

FIG. 3 is a layout of the top of a sensor card. A printed circuit board has a top and a bottom portion. The top portion 300 of the sensor card is shown in FIG. 3. Included in the design are locations for a microcontroller 310, a voltage regulator 312, an accelerometer 314, a real time clock 316, and a crystal 318. The microcontroller 310 may control the operation of the biosensor and the storing and analysis of data collected by the biosensor. The voltage regulator 312 may power down portions of the biosensor when the biosensor is inactive. The accelerometer 314 may obtain data on motion in three axes. The real time clock 316 may be connected to a battery so that proper time may be maintained even when the voltage regulator 312 is powered down. The crystal 318 may be connected to the real time clock to aid in accurately keeping track of the time. The real time clock 316 may be updated when the biosensor is attached to a computer such as through a USB connector.

A battery connection 320 may provide for mounting of a battery or for wires connecting to the battery. A crystal 322 may be connected to the microcontroller 310 to aid in the microcontroller 310 operation at the correct frequency. An OpAmp module 324 may be used as part of the electrodermal activity sensor circuitry. Based on skin contacting electrodes in the biosensor a flip flop 326 may be set to a “1” state. When the flip flop output is set to a “1” state the voltage regulator 312 may be turned on. When the voltage regulator 312 is turned on, the microcontroller 310 and the accelerometer 314 may be powered up. During testing and evaluation of the sensor card, probe points (330-340) may be used to aid in debugging the sensor card.

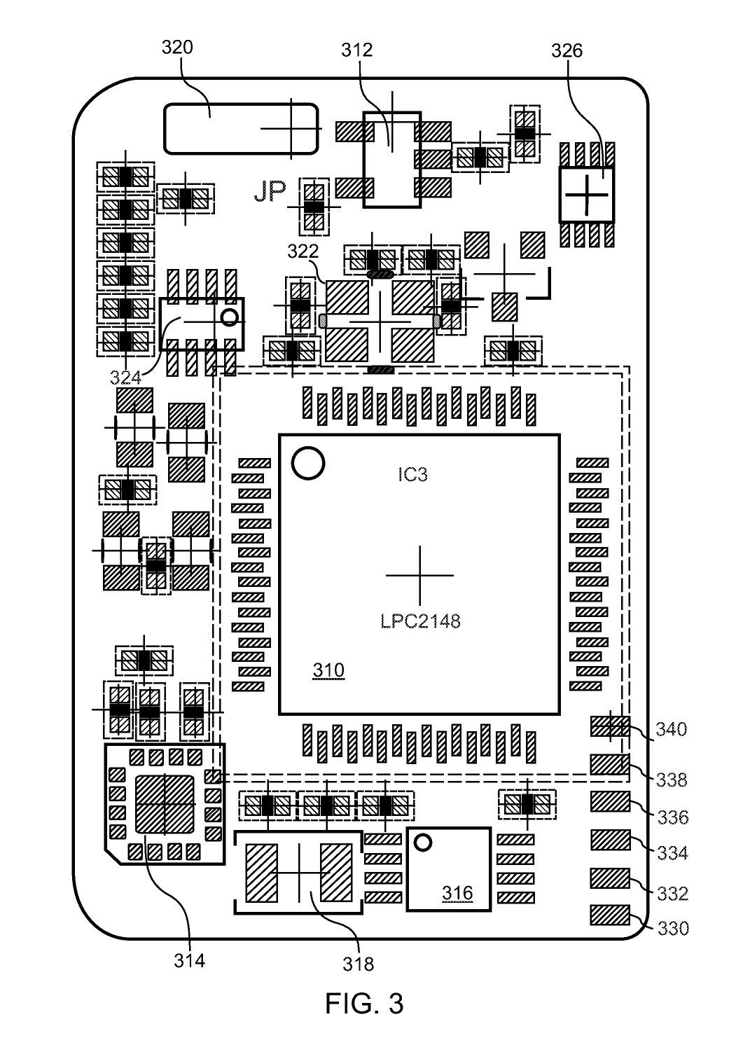

FIG. 4 is a layout of the bottom of a sensor card. The sensor card may include connections for a status LED 410, a charging LED 412, a charging integrated circuit chip 420, a USB connector 430, a comparator 440, a memory card holder 442, and a button 444. The status LED 410 may blink when the biosensor is powered on. The biosensor may be automatically powered on by having skin come in contact with electrodes from the biosensor. The biosensor may also be powered on by pushing button 444. By having either of these events occur, the flip flop output may be set to a “1” state and the voltage regulator 312 may be turned on. The status LED 410 may also blink occasionally to notify the user that the biosensor is still running. The status LED 410 may blink every five seconds while the biosensor is running. The status LED 410 may blink green when more than half of the battery life remains. The status LED 410 may blink yellow when a battery life of 25 to 50% remains. The status LED 410 may blink red when less than 25% of the battery life remains. The status LED 410 may blink blue when an event is marked, such as by pushing the button 444. The charging LED 412 may turn on when the battery is being charged, such as when the biosensor is attached to a USB connector cable through the USB connector 430 to a computer or other device. The charging integrated circuit chip 420 may be used when the USB connector 430 is connected and the battery is being charged. The charging integrated circuit chip 420 controls current flow to the battery for proper charging of the battery.

The comparator 440 evaluates the current flow through the electrodes and compares a voltage on an OpAmp output with a threshold voltage. If the threshold voltage is exceeded on the OpAmp output, there is an indication of electrodermal activity due to skin contacting the electrodes of the biosensor. The threshold voltage may be half of the battery voltage plus 15 mV. The memory card holder 442 may be for connecting an SD memory card or some other form of non-volatile memory. The button 444 may be depressed when an individual wants to turn on the biosensor, turn off the biosensor, or mark an event for recording. It may be desirous to mark an event for later study. An event might be marked at the beginning of an activity, such as viewing a web page, walking through a store, using a product, starting a game, starting exercise, or some other activity of interest.

In an embodiment, the sensor board may be based around an LPC2148 ARM7-TDMI microcontroller by NXP™. The LPC2148 includes on-board analog-to-digital converters (ADCs), USB 2.0 support, and 512 kB of on-board flash for program storage. A microSD secure-digital (SD) card may be utilized for data storage because of its small footprint and ready availability in a variety of flash sizes. The LPC2148 may make use of a Serial-Peripheral Interface (SPI) to communicate with the SD card, and may write log files in FAT16 format for easy interoperation with PC computers. A real time clock chip PCF8563 from NXP™ may be used, allowing it to retain time of day information between uses. The ability to provide timestamps in log files is useful, as time-of-day information provides further context for the data which is used by researchers. A small Lithium-Polymer battery with integrated protection circuit may be used to power the device, and may be automatically recharged when the unit is plugged in via the USB port. A MAX1555 by Maxim IC™ may be used to control battery charging.

In an embodiment, to measure motion information, a MEMS (micro electromechanical system) three-axis accelerometer may be used, such as the ADXL335 by Analog Devices™. For the measurement of electrode temperature, an integrated circuit temperature sensor thermally coupled to the electrode interconnect may be used. In some implementations, thermal epoxy or thermal tape may be used to ensure a low thermal resistance between the sensor and the electrode coupling. In order to provide extra protection against overheating of the sensor circuitry in a failure condition, a digital thermal monitor may also be used.

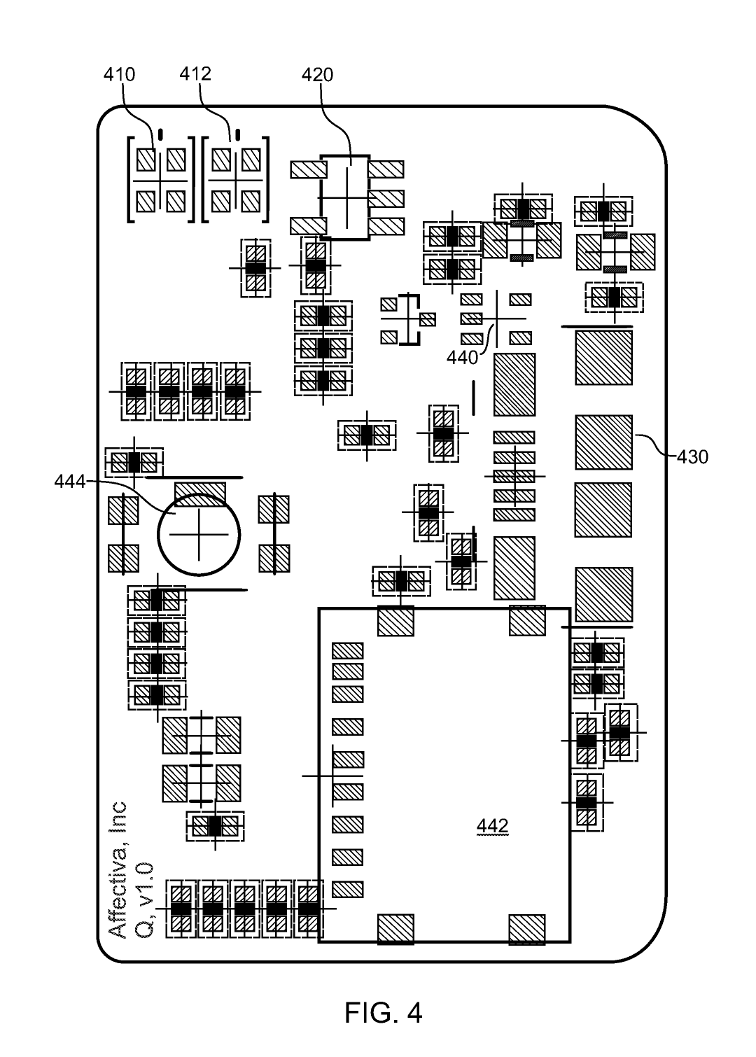

FIG. 5 is a flowchart for automatically powering on physiological sensor. The process 500 begins with contacting the skin with the biosensor 510. The contact may be performed by a plurality of electrodes from the biosensor. There may be two electrodes which contact the skin. A battery may be connected to one of the electrodes. A sensor circuit, which is part of the biosensor, may be connected to the second electrode. A current supplied by the battery may flow from one electrode to the other through the skin of a person. This electrodermal activity indicates that the electrodes are in contact with the person.

The skin conductance, also known as electrodermal activity (EDA), may be sensed 520. The current flowing from one electrode to the other through the skin may produce a voltage variation in a sensing circuit on the biosensor. This voltage variation may be beyond a threshold voltage. Once this threshold voltage is crossed a comparator output may switch to a “1” state. This comparator output may switch a flip flop output to a “1” state.

A voltage regulator output may turn on 530. The flip flop output being set to a “1” state may cause a flip flop output to go to a “1” state. This flip flop output may be an input to the voltage regulator which causes the voltage regulator output to turn on. Various circuitry within the biosensor may be connected to the output of the voltage regulator. Some of the circuitry which is attached to the output of the voltage regulator may be a microcontroller, an accelerometer, a temperature sensor, and LEDs. Some or all of this circuitry may be powered down when the voltage regulator is turned off. Likewise some or all of this circuitry may be powered on when the voltage regulator is turned on. By powering down circuitry battery life may be extended when the biosensor is not in use. By using electrodermal activity to sense when to turn on the biosensor, only a small amount of current is required in the biosensor and thus battery life can be quite long. Further, by using electrodermal activity to determine when to turn on the biosensor, the time duration to power up the biosensor can be quite short. The time duration is driven only by the delay of an OpAmp, a comparator, a flip flop, and a voltage regulator, along with the associated wiring delays. From a human observer's perspective the time to turn on is instantaneous. The voltage regulator may turn on within 5 seconds of the plurality of electrodes contacting the skin. In some embodiments, the delay may be less than a quarter second, less than a half second, less than one second, or less than five seconds. In some other embodiments, circuit configurations may be chosen with delays less than 30 seconds, less than a minute, or less than five minutes.

The process 500 may include powering on a wearable physiological sensor comprising contacting skin of an individual to a pair of electrodes, sensing skin conductance for the individual through the pair of electrodes, turning on an output of a voltage regulator in response to the sensing of the skin conductance based on the contacting of the skin to the pair of electrodes.

In some embodiments, feedback may be provided to the user 540. The feedback may be the turning on or blinking of an LED. The feedback may be a vibration or some other indication. The total time delay to turn on the biosensor may include the time to blink the LED.

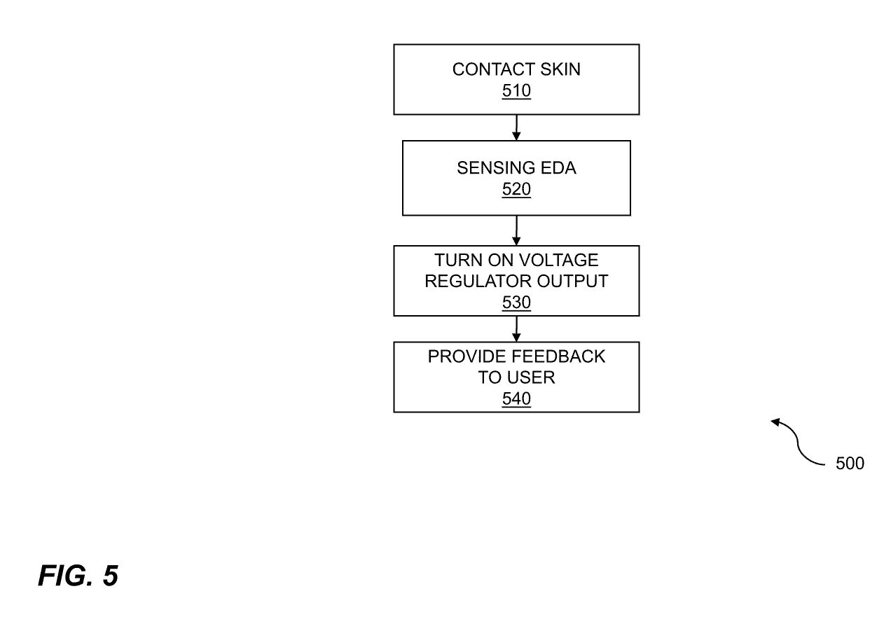

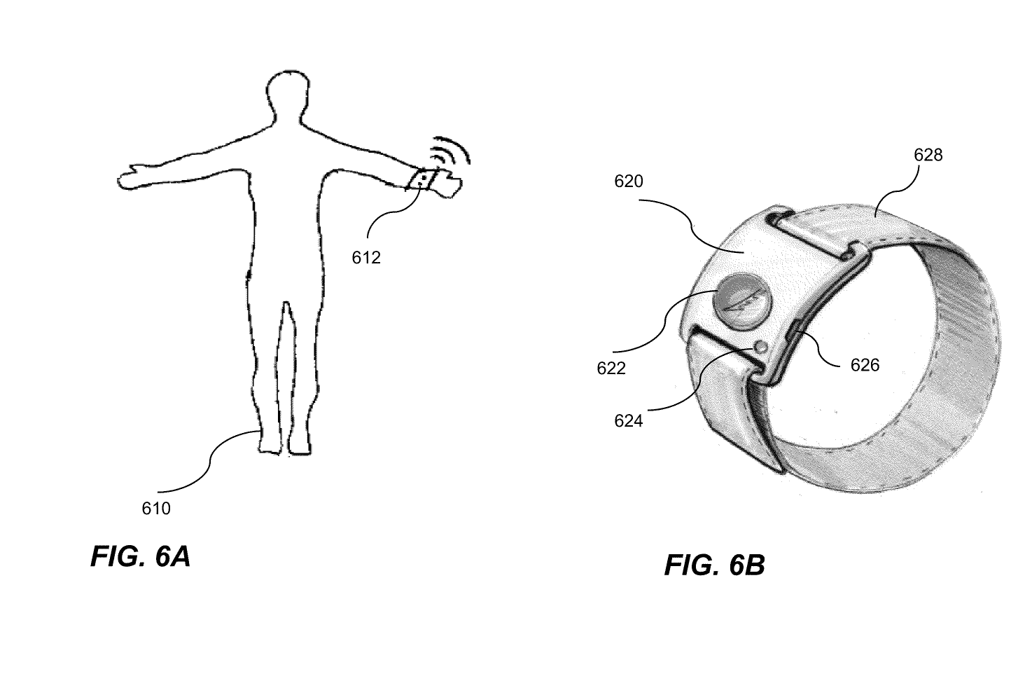

FIG. 6A is a diagram of a sensor attached to an individual. A body 610 for a person is shown. A biosensor 612 may be attached to the body 610. The biosensor 612 may be attached to a wrist, to a hand, or to some other part of the body 610. The biosensor 612 may be attached by a wristband, a sleeve, an adhesive, or some other means. The biosensor 612 may store data collected from the person. The data collected may include electrodermal activity readings, accelerometer readings, skin temperature readings, heart rate, heart rate variability, or other information. All of these readings may be considered physiological data. Alternatively, the accelerometer readings may be considered activity measurements and the electrodermal activity, skin temperature, and heart rate readings may be considered physiological data. The various data collected may later be read through a USB or other port. Alternatively, a wireless connection to the biosensor 612 may be used to continuously or occasionally download the collected data.

FIG. 6B is a drawing of a biosensor with a wristband. A biosensor 620 is shown with a button 622, an LED 624, a USB port 626, and a wristband 628. The biosensor 620 may be used for collecting electrodermal activity readings, accelerometer readings, skin temperature readings, heart rate, heart rate variability, or other information. The biosensor 620 may have two electrodes (not shown) on the side of the biosensor 620 toward the skin. These electrodes may be used to measure electrodermal activity. One or more electrodes may also be used to measure skin temperature. The button 622 may be used to turn off the biosensor 620, turn on the biosensor 620, or to mark an event of interest. By pressing the button 622, the user may record a point of interest with the data being collected. Then later the data can be analyzed recognizing the point of interest marked along with the associated data.

The LED 624 may blink when the biosensor 620 turns on. The LED 624 may also be used to indicate that the biosensor 620 is continuing to operate or the state of the battery. The USB port 626 may be on one of the sides or top or bottom of the biosensor 620. Numerous types of ports may be used including a USB port, a mini-USB port, a micro-USB port or other type of connection. These connections may be used to charge a battery within the biosensor 620 and may be used to store data or instruction on the biosensor 620 or to read data collected by the biosensor 620. The wristband 628 may be used to secure the biosensor to an individual. Velcro may be used to secure the wristband 628. Alternatively, a clasp or other means of attaching the wristband may be used.

The shape of biosensor 620 is shaped in a curve to be adapted for use on a person's wrist. Other shapes or biosensors may be used such as a biosensor which is flat on both the top and bottom side. Other apparatus may be used for securing the biosensor to the person's body. For instance a cloth sleeve may be adapted to fit around a wrist. The cloth sleeve may be formed from a stretchable fabric for ease of use and comfort. The cloth sleeve may be secured around the wrist by Velcro, snaps, a zipper, or buttons. The cloth sleeve may have a pocket within which biosensor fits. The pocket may be securable with a flap that has a snap, zipper, buttons, or Velcro on it. The biosensor may also be adapted to fit another part of the body. The cloth sleeve or other securing device may be adapted to fit the shape of the other part of the body.

It will be understood that for each flow chart, the depicted steps or boxes are provided for purposes of illustration and explanation only. The steps may be modified, omitted, or re-ordered and other steps may be added without departing from the scope of this disclosure. Further, each step may contain one or more sub-steps. While the foregoing drawings and description set forth functional aspects of the disclosed systems, no particular arrangement of software and/or hardware for implementing these functional aspects should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. Each element of the block diagrams and flowchart illustrations, as well as each respective combination of elements in the block diagrams and flowchart illustrations, illustrates a function, step or group of steps of the methods, apparatus, systems, computer program products and/or computer-implemented methods. Any and all such functions may be implemented by computer program instructions, by special-purpose hardware-based computer systems, by combinations of special purpose hardware and computer instructions, by combinations of general purpose hardware and computer instructions, and so on. Any and all of which may be generally referred to herein as a “circuit,” “module,” or “system.”

A programmable apparatus which executes any of the above mentioned computer program products or computer implemented methods may include one or more microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. It will also be understood that a computer may include a Basic Input/Output System (BIOS), firmware, an operating system, a database, or the like that may include, interface with, or support the software and hardware described herein.

Embodiments of the present invention are not limited to applications involving conventional computer programs or programmable apparatus that run them. It is contemplated, for example, that embodiments of the presently claimed invention could include an optical computer, quantum computer, analog computer, or the like. Regardless of the type of computer program or computer involved, a computer program may be loaded onto a computer to produce a particular machine that may perform any and all of the depicted functions. This particular machine provides a means for carrying out any and all of the depicted functions.

Any combination of one or more computer readable media may be utilized. The computer readable medium may be a non-transitory computer readable medium for storage. A computer readable storage medium may be electronic, magnetic, optical, electromagnetic, infrared, semiconductor, or any suitable combination of the foregoing. More specific examples of the computer readable storage medium may include an electrical connection having one or more wires, a portable computer diskette, a hard disk, a random access memory (RAM), a read-only memory (ROM), an erasable programmable read-only memory (EPROM, Flash, MRAM, FeRAM, or phase change memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

It will be appreciated that computer program instructions may include computer executable code. A variety of languages for expressing computer program instructions may include without limitation C, C++, Java, Javascript, assembly language, Lisp, Perl, Tcl, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed more or less simultaneously to enhance utilization of the processor and to facilitate substantially simultaneous functions. By way of implementation, any and all methods, program codes, program instructions, and the like described herein may be implemented in one or more thread. Each thread may spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described.

While the invention has been disclosed in connection with preferred embodiments shown and described in detail, various modifications and improvements thereon will become readily apparent to those skilled in the art. Accordingly, the spirit and scope of the present invention is not to be limited by the foregoing examples, but is to be understood in the broadest sense allowable by law.

What is claimed is:

1. A wearable apparatus for monitoring physiological information for an individual comprising:
a plurality of electrodes for contacting skin on the individual;
a battery powering at least one of the plurality of electrodes;
a sensor for determining skin conductance across the plurality of electrodes; and
a power control that responds to the sensor wherein the sensor determines that the plurality of electrodes are in contact with the skin based on the skin conductance and wherein the power control powers up circuitry when the plurality of electrodes are in contact with the skin.

2. The apparatus of claim 1 wherein the power control includes a voltage regulator.

3. The apparatus of claim 1 wherein the power control includes clock gating.

4. The apparatus of claim 1 wherein the power control includes disabling a microcontroller.

5. The apparatus of claim 1 wherein the plurality of electrodes comprise two electrodes.

6. The apparatus of claim 5 wherein the sensor includes an analog sensing stage to measure conductance between the two electrodes in contact with the skin and wherein the sensor further includes a high-gain transconductance amplifier circuit.

7. The apparatus of claim 6 wherein the conductance which is measured comprises a current value.

8. The apparatus of claim 1 wherein the sensor further comprises an operational amplifier connected to one of the plurality of electrodes wherein the operational amplifier is used to evaluate the skin conductance.

9. The apparatus of claim 8 wherein the sensor further comprises a comparator which determines that a voltage on an output of the operational amplifier has risen above a threshold.

10. The apparatus of claim 2 wherein the voltage regulator turns on within 5 seconds of the plurality of electrodes contacting the skin.

11. The apparatus of claim 1 further comprising real time clock circuitry connected to the battery.

12. The apparatus of claim 2 further comprising a microcontroller, a temperature sensor, an accelerometer, and a storage memory each of which has its power controlled by the voltage regulator.

13. A method for powering on a wearable physiological sensor comprising:
contacting skin of an individual to a pair of electrodes;
sensing skin conductance for the individual through the pair of electrodes; and
turning on an output of a voltage regulator in response to the sensing of the skin conductance based on the contacting of the skin to the pair of electrodes.

14. The method of claim 13 wherein the sensing is accomplished by an analog sensing stage to measure current between the pair of electrodes in contact with the skin and wherein the analog sensing stage further includes a high-gain transconductance amplifier circuit.

15. The method of claim 13 further comprising real time clock circuitry which is not connected to the output of the voltage regulator.

16. The method of claim 13 further comprising a microcontroller, a temperature sensor, an accelerometer, and a storage memory each of which is connected to the output of the voltage regulator.

17. A wearable system for detecting physiological information from an individual comprising:
a pair of electrodes for contacting skin of the individual;
power supply means for powering at least one of the pair of electrodes;
sensing means for determining skin conductance across the pair of electrodes; and
a voltage regulator that responds to the sensing means wherein the sensing means determines that the pair of electrodes is in contact with the skin based on the skin conductance and wherein the voltage regulator powers up circuitry when the pair of electrodes is in contact with the skin.

18. The system of claim 17 further comprising real time clock circuitry connected to the power supply means.
